(19) United States
(12) Patent Application Publication
(10) Pub. No.: US 2020/0000650 A1
(43) Pub. Date: Jan. 2, 2020

(54) INDIVIDUAL PULL TAB PACKAGING FOR SANITARY PADS

(71) Applicant: Laura Bollier, Sandy Springs, GA (US)

(72) Inventor: Laura Bollier, Sandy Springs, GA (US)

(21) Appl. No.: 16/025,905

(22) Filed: Jul. 2, 2018

(51) Int. Cl. A61F 13/551; B65D 77/32; A61F 13/472; A61F 13/84

(52) U.S. Cl. CPC A61F 13/5514; B65D 77/32; A61F 13/472; A61F 2013/8497; A61F 13/84; A61F 2013/55195; A61F 13/5515

(57) ABSTRACT

Implementations of an individual pull tab packaging for sanitary pads are provided. In some implementation the packaging comprises a compacted housing in the shape of a compacted cylinder wherein the height of the housing is smaller than the width of the cylinder. In some implementations, the compacted housing comprises a top portion having a circular shape, a bottom portion having a circular shape, and a cylindrical side extending from the top portion to the bottom portion. In some implementations, the inner surface of the top portion, inner surface of the bottom portion, and inner surface of the cylindrical side define a closed interior configured to hold and enclose a pad. In some implementations, the cylindrical side comprises a separable strip of material configured to be pulled away from a portion of the cylindrical side to access the interior of the compacted housing to remove a pad housed within the housing through an opening.

### TECHNICAL FIELD

This disclosure relates to implementations of individual pull tab packaging for sanitary pads.

### BACKGROUND

FIG. 1A illustrates a standard feminine hygiene product in the form of a sanitary pad and FIG. 1B illustrates typical individual packaging for sanitary pads. A sanitary pad may be carried by a person in their hand, a purse or other handbag, or in a clothing pocket. However, individually carried feminine hygiene products provide little or no discretion or privacy because such products are easily noticeable due to their minimal and recognizably packaging. Furthermore, individually carried feminine hygiene products have little protection from damage such as crushing or tearing, since the products are minimally packaged in paper or thin plastic wrapping as shown in FIG. 1B.

### BRIEF DESCRIPTION OF THE DRAWINGS

FIG. 1A illustrates an example existing unpackaged sanitary pad that can be held and enclosed in implementations of example individual pull tab packaging for sanitary pads according to the present disclosure.

FIG. 1B illustrates an existing example of a minimal packaging for an existing sanitary pad such as the sanitary pad shown in FIG. 1A.

FIGS. 2A-2C illustrate implementations of an example individual pull tab packaging for sanitary pads according to the present disclosure.

FIG. 3A illustrates an example fabrication layout of the individual pull tab packaging for sanitary pads shown in FIGS. 2A-2C.

FIG. 3B illustrates an example fabrication layout of a support tray of the individual pull tab packaging for sanitary pads shown in FIGS. 2A-2C.

FIGS. 4A-4C illustrate example folding configurations of a sanitary pad that can be held and enclosed in the individual pull tab packaging for sanitary pads shown in FIGS. 2A-2C.

### DETAILED DESCRIPTION

Implementations of an individual pull tab packaging for sanitary pads are provided. In some implementations, the individual pull tab packaging for sanitary pads comprises a hollow compacted cylinder configured to hold and enclose a sanitary pad within an interior and comprising a pull tab to be pulled away from the compacted cylinder to access the interior and removing a sanitary pad housed within the compacted cylinder through an opening.

In some implementations, a method for using the individual pull tab packaging for sanitary pads comprising pulling the pull tab away from the cylindrical side to access the interior of the compact cylinder and removing an enclosed sanitary pad from the interior through the opening.

In some implementations, the individual pull tab packaging for sanitary pads is configured to have an external appearance that looks like a makeup or cosmetics container. For example, in some implementations, the individual pull tab packaging for sanitary pads is configured to have an external appearance that looks like a makeup or cosmetics compact, such as a powder compact or a mirror compact. For example, in some implementations, the packaging for sanitary pads is a definite or fixed hollow compacted cylinder similar to the case for blush press powder, loose powder, foundation, etc. or a mirror compact etc. In some implementations, the packaging for sanitary pads has a top piece and bottom piece that form a hollow compacted cylinder where the top piece and bottom piece are separable by any manner. In this way, in some implementations, the individual packaging for sanitary pads provides discretion and/or privacy for carrying individual sanitary pads on-person, such as in a purse, pocketbook, or other handbag or in a clothing pocket.

In some implementations, the individual pull tab packaging for sanitary pads is configured to have a sanitary or sterile interior to hold and enclose a sanitary pad without the need for any additional packaging enclosing the sanitary pad within the interior, such as an unpackaged sanitary pad. In this way, in some implementations, the individual pull tab packaging for sanitary pads provides a sanitary packaging to hold and enclose a sanitary pad, including an unpackaged sanitary pad.

In some implementations, the individual pull tab packaging for sanitary pads is composed of cardboard or other similar material to hold and enclose a sanitary pad. In this way, in some implementations, the individual pull tab packaging for sanitary pads provides a protective packaging from damage such as crushing or tearing for an unpackaged or minimally packaged sanitary pad, such as in paper or thin plastic wrapping, that is carried on-person, such as in a purse, pocketbook, or other handbag or in a clothing pocket.

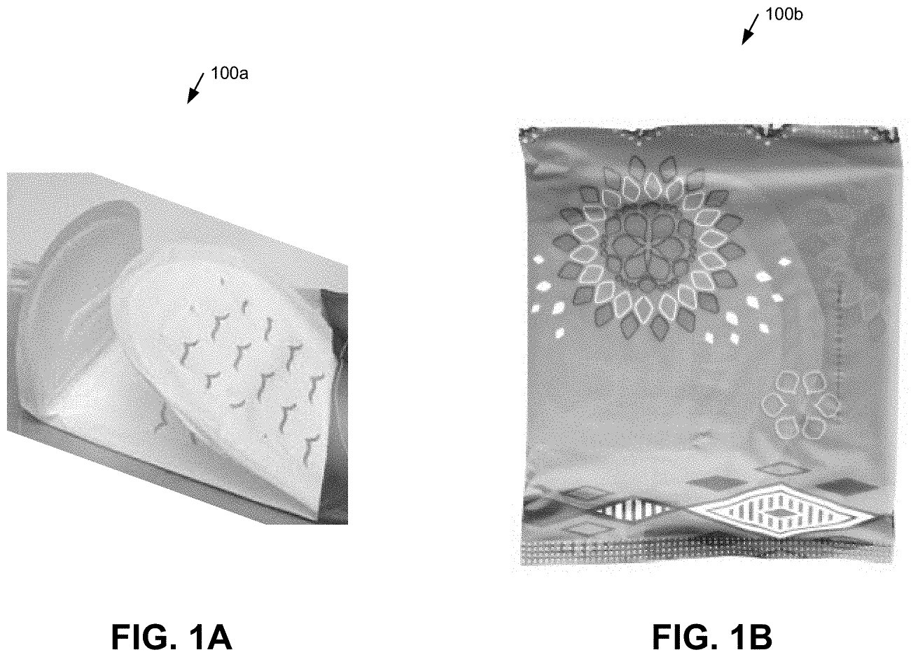

FIG. 1A illustrates an example existing unpackaged sanitary pad 100a that can be held and enclosed in implementations of example individual pull tab packaging for sanitary pads according to the present disclosure. Feminine hygiene products, such as the sanitary pad 100a, are carried individually on-person, such as in a purse, pocketbook, or other handbag or in a clothing pocket. However, such individually carried feminine hygiene products 100a are easily noticeable and provide little or no discretion or privacy, since such products are either unpackaged or minimally and recognizably packaged (e.g., as shown in FIG. 1B).

FIG. 1B illustrates an existing example of a minimal packaging 100b for an existing sanitary pad such as the sanitary pad 100a shown in FIG. 1A. Such minimal packaging 100b is usually a paper or thin plastic wrapping. Such minimal packaging 100b is usually recognizable and/or provides little or no discretion or privacy. Such minimal packaging 100b provides little or no protection to a sanitary pad 100a from damage such as crushing or tearing when carried on-person, such as in a purse, pocketbook, or other handbag or in a clothing pocket.

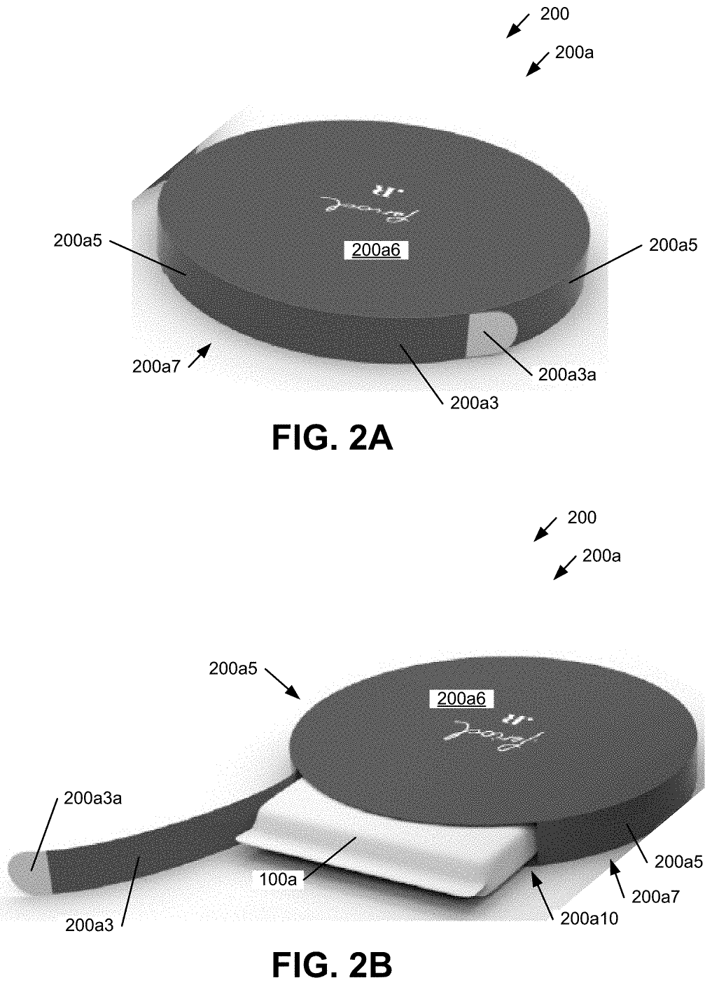

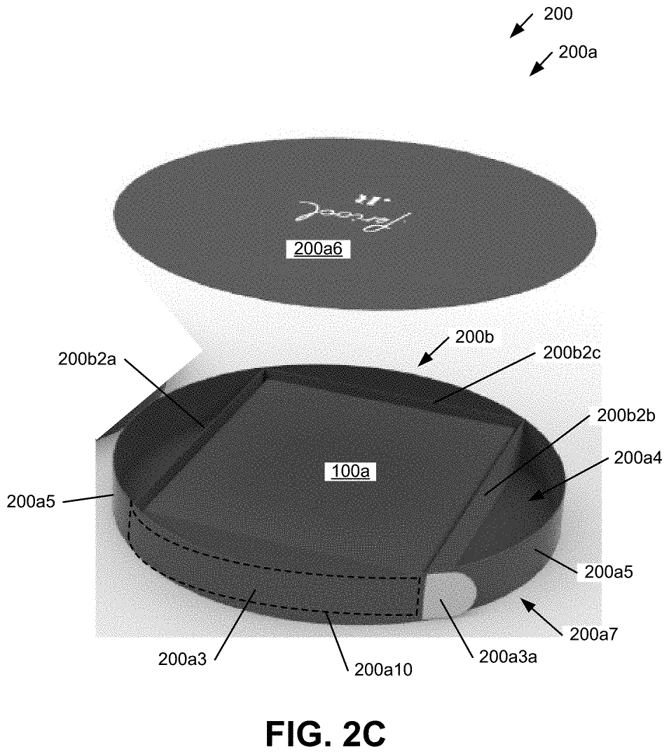

FIGS. 2A-2C illustrate implementations of an example individual pull tab packaging for sanitary pads 200 according to the present disclosure. In some implementations, the individual pull tab packaging for sanitary pads 200 comprises a hollow compacted cylinder 200a that comprises a top base 200a6, a bottom base 200a7, and a cylindrical surface 200a5 that extends between the top base 200a6 and the bottom base 200a7 where the height of the cylinder is smaller than the diameter of the cylinder. In some implementations, the compacted cylinder 200a is configured to have an external appearance that looks like a makeup container or cosmetics container. For example, in some implementations, the cylinder 200a is configured to have an external appearance that looks like a makeup or cosmetics compact, such as a powder compact or a mirror compact.

The cylindrical surface 200a5 extending between the top base 200a6 and the bottom base 200a7 comprises a pull tab 200a3 configured to access an interior 200a4 of the cylinder 200a when the pull tab 200a3 is pulled away from the remainder of the cylindrical surface 200a5.

As shown in FIG. 2C, in some implementations, the cylinder 200a is configured to hold and enclose a sanitary pad 100a in the interior 200a4 of the cylinder 200a. For example, in some implementations, the cylinder 200a is configured to house and store a sanitary pad 100a in the sealed or otherwise closed interior 200a4 of the cylinder 200a.

In some implementations, the interior 200a4 of the cylinder 200a is defined by an inner surface of the top base 200a6, an inner surface of the bottom base 200a7, and an inner surface of the cylindrical surface 200a5 that extends between the top base 200a6 and the bottom base 200a7. In some implementations, the interior 200a4 is sanitary, sterile, or otherwise uncompromised to hold and enclose the sanitary pad 100a without any additional packaging enclosing the sanitary pad 100a within the interior 200a4. In some implementations, the sanitary pad 100a may comprise additional packaging.

In some implementations, as discussed above, the pull tab 200a3 is configured to access an interior 200a4 of the cylinder 200a when the pull tab 200a3 is pulled away from the remainder of the cylindrical surface 200a5. In some implementations, the pull tab 200a3 is a portion of the cylinder 200a that is a piece or strip of material that is separable from a portion of the cylindrical surface 200a5. In some implementations, the pull tab 200a3 extends around a portion of the outer surface of the cylinder 200a. In some implementations, the pull tab 200a3 is configured to be pulled from a portion of the cylindrical surface 200a5 to access the interior 200a4 of the cylinder 200a through an opening 200a10 of the cylinder 200a. In some implementations, the opening 200a10 is sized to pass a sanitary pad 100a through the opening to remove/insert the sanitary pad 100a from/in the interior 200a4. In some implementations, the pull tab 200a3 includes a distal end portion 200a3a′ configured to be pulled to separate the pull tab 200a3 from a portion of the cylindrical surface 200a5.

In some implementations, the bottom base 200a7 is flat. In some implementations, the top base 200a6 is flat.

In some implementations, at least a portion of the cylindrical surface 200a5 is perforated between the top base 200a6 and a first side of the pull tab 200a3. In some implementations, the cylindrical surface 200a5 is perforated between the bottom base 200a7 and a second side of the pull tab 200a3. In this way, in some implementations, the pull tab 200a3 can be torn away from away from the remainder of the cylindrical surface 200a5 to access the interior 200a4 of the cylinder 200a.

In some implementations, at least a portion of the pull tab 200a3 is adhesively attached a portion of the cylindrical surface 200a5. In some implementations, the adhesive attachment is configured to allow the pull tab 200a3 to be pulled away from the remainder of the cylindrical surface 200a5 to access the interior 200a4 of the cylinder 200.

In some implementations, the pull tab 200a3 is attached to the cylindrical surface 200a5 in any other suitable manner that allows the pull tab 200a3 to be pulled away from the remainder of the cylindrical surface 200a5 to access the interior 200a4 of the cylinder 200.

In some implementations, the opening 200a10 of the cylinder 200a is resealable or otherwise recloseable with the pull tab 200a3 so that the cylinder 200a can be used to store or discard a sanitary pad 100a (e.g., after it is used).

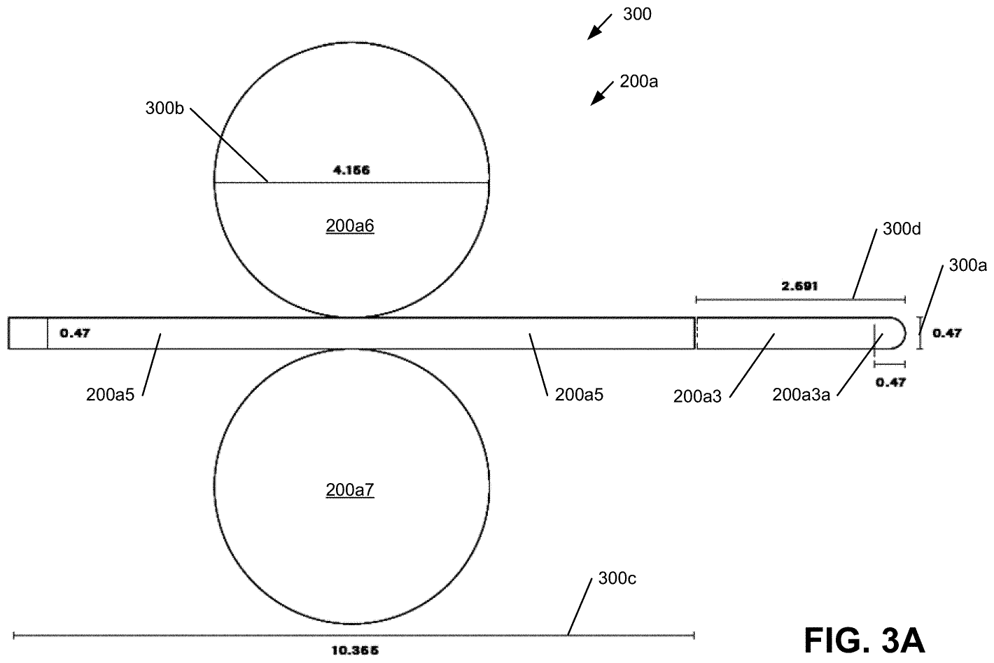

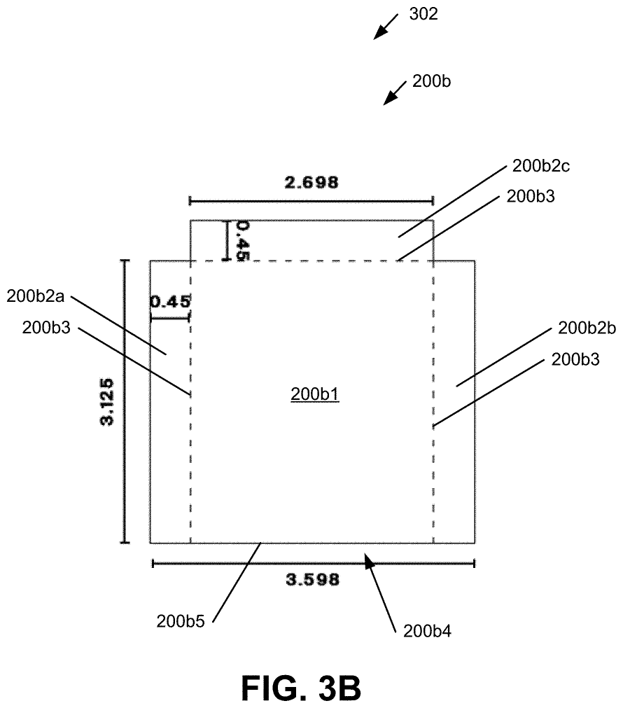

Referring to FIGS. 2A-2C and FIG. 3B (which illustrates a fabrication layout discussed further below), in some implementations, the individual pull tab packaging for sanitary pads 200 further comprises a support tray 200b housed within the interior 200a4 of the cylinder 200a. In some implementations, the support tray 200b is configured to position a sanitary pad 100a within the interior 200a4. In some implementations, the support tray 200b is configured to guide a sanitary pad 100a out of and/or into the interior 200a4 of the cylinder 200a.

In some implementations, the support tray 200b comprises a base 200b1 that positions upon the bottom base 200a7 of the cylinder 200a. In some implementations, the base 200b1 is a portion of the inner surface of the bottom base 200a7. In other words, in some implementations, the support tray 200b has no base and only sides. In some implementations, the support tray 200b comprises a plurality of sides 200b2 extending from a respective plurality of edges 200b3 of the support tray base 200b1. As shown in FIG. 2C, in some implementations, a first side 200b2a of the support tray 200b extends from (or is near or adjacent to) a first side of the opening 200a10 and a second side 200b2b of the support tray 200b extends from (or is near or adjacent to) a second side of the opening 200a10. In some implementations, the sides 200b2 are configured to guide the sanitary pad out of and/or into the interior of the cylinder upon the support tray base 200b1. In some implementations, the sides 200b2 are configured to position the sanitary pad 100a within the interior 200a4 upon the support tray base 200b1.

In some implementations, the support tray 200b comprises an open side 200b4 along another edge 200b5 of the support tray base 200b1. In some implementations, the open side 200b4 is configured to align with the opening 200a10 of the cylinder 200a to allow the sanitary pad 100a to be removed from the interior 200a4 off of the support tray base 200b1. In some implementations, the open side 200b4 is configured to align with the opening 200a10 of the cylinder 200a to allow the sanitary pad 100a to be inserted into the interior 200a4 upon the support tray base 200b1.

FIG. 3A illustrates an example fabrication layout or pattern 300 of the individual pull tab packaging for sanitary pads 200 shown in FIGS. 2A-2C without the support tray. FIG. 3A illustrates the cylindrical surface 200a5 in a flattened position, the the top base 200a6, and bottom base 200a7 prior to fabrication to make the packaging 200. In some implementations, the fabrication layout 300 also includes example dimensions in inches of the components of the packaging 200, such as the height 300a of the cylinder 200a, the width 300b of the cylinder 200a (which is also the diameter 300b of the top base 200a6 and the bottom base 200a7), the length 300c of the cylindrical surface 200a5 not including the pull tab 200a3, and the length 300d of the pull tab. As shown in FIG. 3A, in some implementations, the width 300b of the cylinder 200a is greater than the height 300a of the cylinder 200a. In some implementations, the cylinder 200a is sized to fit inside of a handbag, such as a purse or a pocketbook, or inside of a clothing pocket. In some implementations, the individual pull tab packaging for sanitary pads 200 comprises any other suitable dimensions, such as the dimensions indicated in FIGS. 3A and 3B. In some implementations, one or more of the components of the packaging 200 comprises dimensions greater than the dimensions indicated in FIG. 3A. In some implementations, one or more of the components of the packaging 200 comprises dimensions less than the dimensions indicated in FIG. 3A.

FIG. 3B illustrates an example fabrication layout or pattern 302 of the support tray 200b of the individual pull tab packaging for sanitary pads 200 shown in FIGS. 2A-2C. FIG. 3B illustrates the support tray 200b in a flattened position prior to fabrication to make the support tray 200b. In some implementations, the fabrication layout 302 includes components of the support tray 200b shown in and/or described above for FIGS. 2A-2C and 3B. In some implementations, the fabrication layout 302 also includes example dimensions in inches of the components of the support tray 200b. In some implementations, one or more of the components of the support tray 200b comprises dimensions less than the dimensions indicated in FIG. 3B. In some implementations, one or more of the components of the support tray 200b comprises dimensions greater than the dimensions indicated in FIG. 3B.

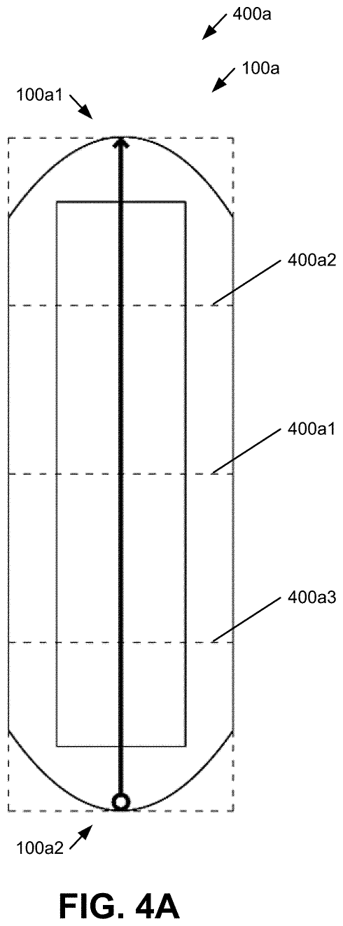

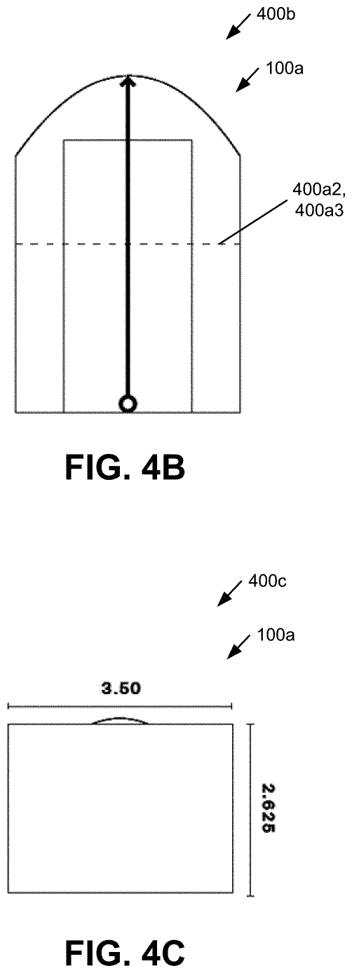

FIGS. 4A-4C illustrate example folding method 400a-c of a sanitary pad 100a that can be held and enclosed in the individual pull tab packaging for sanitary pads 200 shown in FIGS. 2A-2C. FIG. 4A illustrates the sanitary pad in a fully unfolded configuration 400a. In some implementations, the pad 100a is folded along fold line 400a1, 400a2, and 400a3. In some implementations, fold line 400a1 is at the middle of the pad. In some implementations, fold line 400a2 is at a mid-way point between a first end 100a1 of the pad and the middle of the pad 400a1. In some implementations, fold line 400a3 is at a mid-way point between a second end 100a2 of the pad and the middle of the pad 400a1.

In some implementations, the pad 100a is first folded along fold line 400a1. FIG. 4B illustrates the sanitary pad 100a in a partially folded configuration 400b after the pad has been folded along fold line 400a1. As shown in FIG. 4B, fold lines 400a2, 400a3 overlap after the first fold. The pad 100a then is folded along fold lines 400a2, 400a3. FIG. 4C illustrates the sanitary pad 100a in a fully folded configuration 400c after the pad has been folded along fold lines 400a2, 400a3.

FIG. 4C illustrates the sanitary pad 100a that is sized to fit through the opening 200a10 and within the interior 200a4 of the cylinder 200a. In some implementations, the interior 200a4 of the cylinder 200a is sized to contain the sanitary pad 100a, for example in the fully folded configuration 400c described above for FIG. 4C.

In some implementations, the individual pull tab packaging for sanitary pads 200 is composed of any suitable materials. For example, in some implementations, the packaging 200 is composed of cardboard or similar material.

In some implementations, the individual pull tab packaging for sanitary pads 200 can have any suitable appearance. For example, in some implementations, as discussed above, the packaging 200 can have an external appearance that looks like a makeup or cosmetics container such as a powder compact or a mirror compact.

In some implementations, an example method of using the individual pull tab packaging for sanitary pads 200 comprises pulling the pull tab 200a3 away from the cylinder 200a to access the interior 200a4 of the cylinder 200a. In some implementations, the method comprises removing the sanitary pad 100a from the interior 200a4 of the cylinder 200a through the opening 200a10 of the cylinder 200a.

In some implementations, the method further comprises inserting a sanitary pad 100a into the interior 200a4 of the cylinder 200a through the opening 200a10 of the cylinder 200a (e.g., after the sanitary pad 100a has been used). In some implementations, the method further comprises covering the opening 200a10 of the cylinder 200a with the pull tab 200a3 to reclose the opening 200a10 to store or discard the a sanitary pad 100a.

In some implementations, the interior of the hollow compacted cylinder may be accessed in any suitable manner other than a pull tab.

The figures, including photographs and drawings, comprised herewith may represent one or more implementations of the individual pull tab packaging for sanitary pads.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. An individual pull tab packaging for sanitary pads comprising
a compacted housing in the shape of a compacted cylinder wherein the height of the housing is smaller than the width of the cylinder, the compacted housing comprising a top portion having a circular shape, a bottom portion having a circular shape, and a cylindrical side extending from the top portion to the bottom portion wherein the inner surface of the top portion, inner surface of the bottom portion, and inner surface of the cylindrical side define a closed interior wherein the closed interior of the packaging is configured to hold and enclose a pad, further wherein:
the cylindrical side extending from the top portion to the bottom portion comprises a separable strip of material configured to be pulled away from a portion of the cylindrical side; and
the packaging is configured such that when the separable strip of material is pulled away from a portion of the cylindrical side, the interior of the compacted housing is accessible to remove a pad housed within the housing through an opening wherein the opening is sized to pass the sanitary pad through the opening.

2. The individual pull tab packaging for sanitary pads of claim 1 further comprising the sanitary pad held and enclosed with the interior of the cylinder.

3. The individual pull tab packaging for sanitary pads of claim 1 further comprising a support tray within the interior of the cylinder, and configured to position the sanitary pad within the interior and guide the sanitary pad as it pass through the opening.

4. The individual pull tab packaging for sanitary pads of claim 1 wherein the compacted housing is configured to have an external appearance that looks like a powder compact or a mirror compact.

5. The individual pull tab packaging for sanitary pads of claim 1 at least a portion of the cylindrical side is perforated between the top portion and a first side of the separable strip of material and perforated between the bottom portion and a second side of the separable strip of material to allow the separable strip of material to be torn away from a portion of the cylindrical side to access the closed interior of the packaging.

6. The individual pull tab packaging for sanitary pads of claim 1 wherein at least a portion of the separable strip of material is adhesively attached a portion of the cylindrical surface, wherein the adhesive attachment is configured to allow the separable strip of material to be pulled away from a portion of the cylindrical side to access the closed interior of the packaging.

7. The individual pull tab packaging for sanitary pads of claim 1 wherein the closed interior of the packaging is sterile to hold and enclose a pad without any additional packaging enclosing the tampon within the interior.

8. The individual pull tab packaging for sanitary pads of claim 1 wherein the opening of the cylinder is recloseable with separable strip of material.

9. The individual pull tab packaging for sanitary pads of claim 1 wherein the bottom portion is flat, and the top portion is flat.

10. The individual pull tab packaging for sanitary pads of claim 1 wherein the compact housing is composed of cardboard.

11. A method of using the individual pull tab packaging for sanitary pads of claim 2, the method comprising:
pulling the separable strip of material away from a portion of the cylindrical side to access the interior of the compact housing; and
removing the sanitary pad from the interior of the cylinder through the opening.

12. The method of claim 11 wherein the opening of the cylinder is recloseable with the pull tab, the method further comprising:
inserting a sanitary pad into the interior of the compact housing through the opening; and
covering the opening with the separable strip of material to reclose the opening to store or discard the sanitary pad.

13. An individual packaging for sanitary pads comprising
a compacted housing in the shape of a compacted cylinder wherein the height of the housing is smaller than the width of the cylinder, the compacted housing comprising a top portion having a circular shape, a bottom portion having a circular shape, and a cylindrical side extending from the top portion to the bottom portion wherein the inner surface of the top portion, inner surface of the bottom portion, and inner surface of the cylindrical side define a closed interior wherein the closed interior of the packaging is configured to hold and enclose a pad, further wherein:
the interior of the compacted housing is accessible to remove a pad housed within the housing through an opening wherein the opening is sized to pass the sanitary pad through the opening; and
a sanitary pad is held and enclosed with the interior of the cylinder.
